(19) United States
(12) Patent Application Publication
(10) Pub. No.: US 2012/0130462 A1
(43) Pub. Date: May 24, 2012

(54) Implantable Lead Comprising an Elongate Lead Body

(75) Inventors: Heinrich Buessing, Berlin (DE); Timo Frenzel, Berlin (DE)

(73) Assignee: BIOTRONIK SE & CO. KG, Berlin (DE)

(21) Appl. No.: 13/293,728

(22) Filed: Nov. 10, 2011

Related U.S. Application Data
(60) Provisional application No. 61/416,778, filed on Nov. 24, 2010.

(51) Int. Cl. A61N 1/05

(52) U.S. Cl. 607/116

(57) ABSTRACT

An implantable lead including an elongate lead body and a functional lead which extends in the longitudinal direction of the lead body and enables the implementation of a medical function of the lead, wherein, in addition to the functional lead and insulated therefrom, a plurality of inductive resistance circuit elements are embedded in the lead body, which reduce a coupling of the functional lead with an external alternating magnetic field or dampen the transmission of electrical high-frequency energy along the lead.

### RELATED APPLICATION

This patent application claims the benefit of co-pending U.S. Provisional Patent Application No. 61/416,778, filed on Nov. 24, 2010, which is hereby incorporated by reference in its entirety.

### TECHNICAL FIELD

The invention relates to implantable leads and, more particularly, to an implantable lead comprising an elongate lead body and a functional lead which extends in the longitudinal direction of the lead body and enables the implementation of a medical function of the lead. Leads of that type are, in particular, stimulation electrode leads (also referred to simply as “electrodes”) of cardiac pacemakers or shock electrode leads of implantable defibrillators, or they can be catheters that contain an elongate conductive structure.

### BACKGROUND

Medical implants, such as, for example, the aforementioned pacemakers and defibrillators, often include an electrical connection to the inside of the patient's body. A connection of this type is used to measure electrical signals or stimulate cells of the body. This connection is often designed as an elongate electrode. Currently, electrical signals are transmitted between the implant and the electrode contacts (e.g., tip, rings, HV shock helixes, sensors, etc.) using materials having good electrical conductivity.

If a system comprised of an implant and an electrode is exposed to strong interference fields (e.g., EMI, MRI, etc.), unwanted consequences can occur, especially a heating-up of parts of the system or electrical malfunctions (e.g., resets). The heating can result in damage to bodily tissue or organs if the heated parts have direct contact with the tissue. This is typically the case with the electrode tip, in particular.

The unwanted malfunction is caused by the interaction of the field with the elongate lead structure of the electrode. The electrode functions as an antenna and receives energy from the surrounding fields. The antenna can dissipate this energy on the leads, which are used for therapeutic purposes, distally into the tissue via the electrode contacts (e.g., tip, ring, etc.), or proximally into the implant.

The same problems also occur with other elongate conductive structures, the proximal end of which is not necessarily connected to an implant (e.g. catheters, temporary electrodes, stents, etc.).

Shielded electrodes are known. The shielding of the electrode mainly counteracts electrical fields that are coupled in from the outside. In addition, these shieldings provide only a particular shielding strength and are stable over the long term when they have an appropriate shield strength. A compromise must therefore be found between increasing the diameter of the electrode—which would have a corresponding effect on the costs and handling of the electrode—and a diminished shielding effect.

To prevent interferences by magnetic alternating fields, especially in magnetic resonance imaging (MRI) apparatuses, and especially to limit the heating of the electrode tip in fields of this type, it was proposed in U.S. Publication No. 2008/0243218 to provide a protective conductor in an electrode lead that turns back on itself in the longitudinal direction. This “billabong” principle likewise utilizes mutual inductances to diminish induced currents. In this case, however, the three-layered helical winding is likewise expected to increase the diameter of the electrode. Moreover, the electrode will have lower conductivity.

From Ladd M., Quick H.: Reduction of Resonant RF Heating in Intravascular Catheters Using Coaxial Chokes, Magnetic Resonance in Medicine, 2000, measures are known for protecting against the heating of intravascular catheters, which is induced by RF resonances, the measures being designed as external protective throttles (referred to as “chokes”). Chokes of that type are situated on the outer sleeve of the electrode and counteract surface currents. However, this solution does not reduce currents that couple into the inner helix. In addition, the electrode diameter is expected to be increased, with the aforementioned consequences.

The present invention is directed toward overcoming one or more of the above-mentioned problems

It is an object of the invention to provide an improved implantable lead of the type described initially that has improved properties in strong external electromagnetic alternating fields and has a simple design, thereby enabling it to be realized in a cost-effective manner.

### SUMMARY

This object is solved by an implantable lead having the features of the independent claim(s). Advantageous developments of the inventive idea are the subject matter of the dependent claims.

A main idea of the invention is to reduce the influence of strong external fields by embedding a plurality of additional conductive elements in the implantable lead. The additional leads (also referred to here as inductive resistive circuit elements or field-decoupling lead elements), which are insulated against the functional lead and function as local mutual inductances, in particular, change the interaction between the external field and the implantable lead in a manner such that a different current distribution forms on the implantable lead. The additional leads reduce a coupling of the functional lead with an external alternating magnetic field and increase the damping of electrical high-frequency energy that is transported along the implantable lead. The unwanted antenna properties of the lead change as a result of this detuning. This results in reduced heating of the distal lead contacts. This advantage applies for various geometric shapes and various positions of the lead, as will be appreciated by one skilled in the art.

The inductive resistive circuit elements can contain, e.g., a nickel-cobalt alloy and, in particular, MP35N®.

In one embodiment of the invention, the inductive resistive circuit elements are designed as rings disposed in a row and interspaced in the longitudinal direction of the lead body. Small rings of that type are available at very low cost. In alternative embodiments, the field-decoupling lead elements are designed as wire loops or windings disposed in a row and interspaced in the longitudinal direction of the lead body. The aforementioned rings, which generate mutual inductance, can also be comprised of ring segments interconnected in a conductive manner.

According to a further embodiment of the invention, the field-decoupling lead elements are distributed evenly along the length of the lead body, in particular being disposed equidistantly from each other. In this case, the distances between the functional lead elements can be smaller than their diameter, in particular. Arranging the field-decoupling lead elements in a row at relatively short distances apart from one another, as described above, results in a continuous effect of the inductive field-decoupling along the entire length and in practically any feasible bending state of the lead.

According to a further embodiment, the field-decoupling lead elements are placed in preformed recesses in the lead body. Corresponding grooves can be formed relatively easily in the plastic or silicone lead body and ensure that the lead elements retain their even spacing in rows even under the influence of relatively strong mechanical loads of the type that can occur, e.g., during implantation or repositioning.

According to an embodiment of the lead, according to the invention, which is particularly significant for practical application, the lead comprises a first and a second functional lead which extend coaxially relative to one another. The field-decoupling lead elements are disposed in the radial direction between the first and the second functional lead, and insulated by the first or the second functional lead.

While, according to the aforementioned embodiments, individual short field-decoupling leads are used to protect the functional lead or leads, the use of relatively elongate lead elements is feasible in other embodiments. According to a further embodiment, the field-decoupling lead elements therefore comprise a helix, which is a subsection of the above-described helical functional lead and extends in the longitudinal direction of the lead body, the turns of which do not have contact with each another. The insulated turns are interconnected along the length of the lead body by electrically connecting longitudinal wires which extend in the direction of the longitudinal axis of the lead body. According to a modification of the latter embodiment, the insulated turns of the aforementioned helix of the functional lead are provided as the field-decoupling lead elements, the helix being formed by at least one connecting wire which is slanted relative to the longitudinal direction of the lead body. The connecting wire extends along at least one subsection of the lead body and connects at least two adjacent turns of the helix. According to a further embodiment, the connecting wires are subsections of a wire helix that extends contradirectionally to the helix of the functional lead.

Preferably, the helix of the functional lead can be designed as a strip or a wire.

Various other objects, aspects and advantages of the present invention can be obtained from a study of the specification, the drawings, and the appended claims.

### DESCRIPTION OF DRAWINGS

Advantages and useful features of the invention also result from the description of special embodiments, below, and with reference to the figures. They show:

FIG. 1 is a schematic diagram which serves to explain the invention;

FIGS. 2A and 2B are schematic representations of an embodiment of the implantable lead according to the invention, in the longitudinal direction (FIG. 2A) and in a cross-sectional view (FIG. 2B);

FIGS. 3A-3C are schematic depictions of a further embodiment of the lead according to the invention;

FIGS. 4A-4C are schematic depictions of a further embodiment which has been modified relative to FIGS. 3A-3C;

FIG. 5 is a schematic longitudinal sectional view of a section of a further lead according to the invention, and

FIG. 6 is a schematic longitudinal sectional view of a section of a further lead according to the invention.

### DETAILED DESCRIPTION

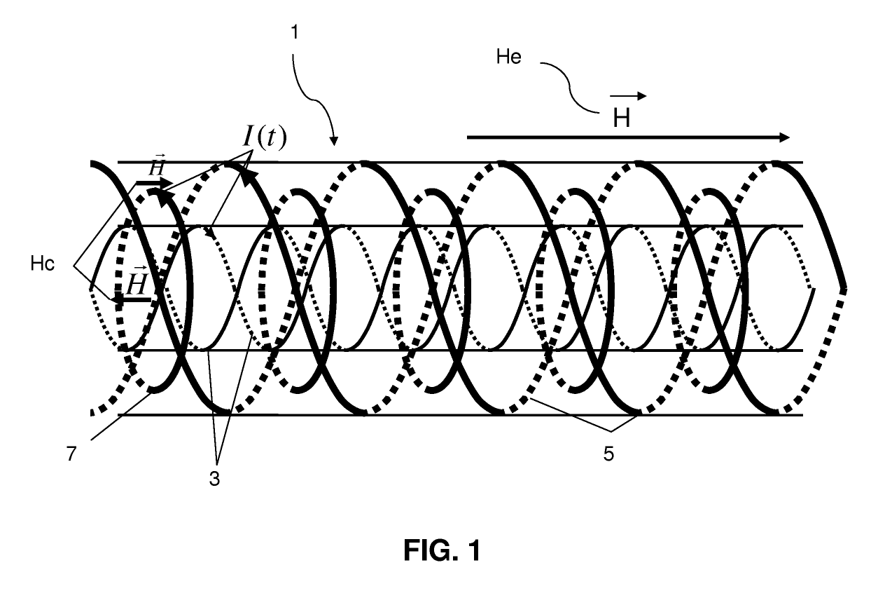

FIG. 1 is a schematic depiction which serves to explain the invention and shows, for clarity, only the conductive elements of an electrode lead 1 as an embodiment of an implantable lead, but not its insulating lead body. In the embodiment shown, an inner conductor 3 which is comprised of a plurality of interwoven wires (also referred to simply as the “first functional lead”), and an outer conductor 5 which is likewise formed of a plurality of interwoven wires (referred to as the “second functional lead”) are provided. In this embodiment, the inner conductor 3 and the outer conductor 5 are wound in opposite directions. However, they may be wound in the same direction. During use, they can be exposed to an external alternating magnetic field Hₑ which induces a current flow I(t) in the conductors 3 and 5.

To reduce disadvantageous influences of these induced currents, conductive rings (which are also referred to as “induction rings” or “field-decoupling lead elements”) 7 disposed equidistantly from one another are situated in the intermediate space between the inner conductor 3 and the outer conductor 5 (i.e., the first and the second functional lead). Due to the time-dependent current flow and the electrical resistance induced therein, rings 7 generate a compensating magnetic field Hₑ which at least partially compensates for the effect of the external magnetic field Hₑ or for electrical losses which diminish the transmission of electrical energy along the conductor.

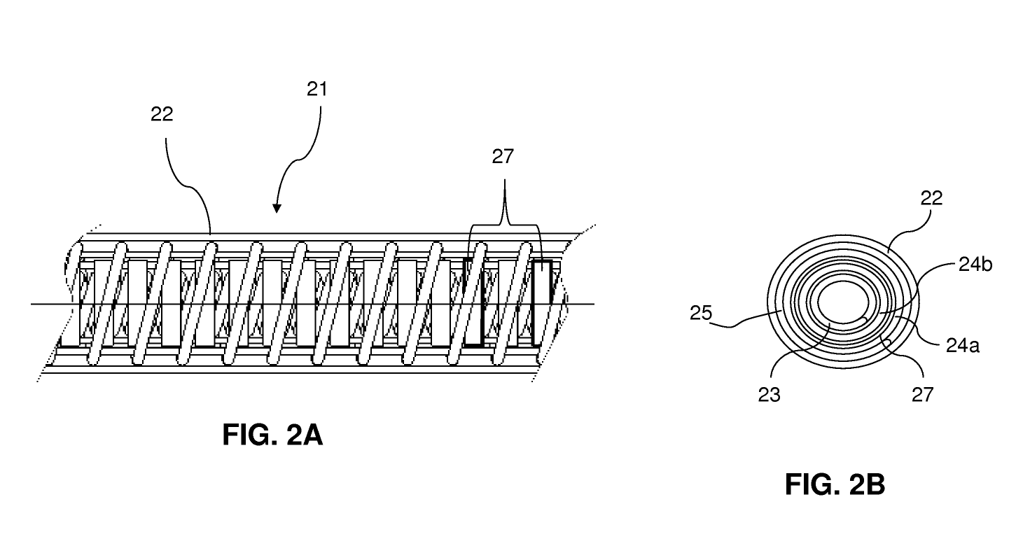

FIGS. 2A and 2B show, in somewhat greater detail, the structural design of a lead 21 according to the invention. In FIGS. 2A-2B, elements that correspond to those shown in the schematic representation in FIG. 1 are labeled with the same reference numbers used therein. Lead 21 comprises, embedded in a lead body 22 (which is depicted schematically in this case) and being insulated from each other via an inner insulating sleeve 24a and a middle insulating sleeve 24b, an inner helix 23 (which itself comprises a plurality of interwoven helixes), an outer helix 25, and induction rings 27 disposed there between. Although these figures are not dimensionally true representations, FIG. 2B does illustrate that the thickness of induction rings 27 is substantially smaller than that of functional leads 23 and 25.

In terms of the geometry of the helixes and the functional leads, and of the induction rings or sleeves, an explanation will be presented on the basis of the example of a special electrode lead which is known as a Setrox electrode. It includes a helix comprised of four wires having a diameter of 0.13 mm. The mean diameter dᵢ of the helix is 0.57 mm. The current Iᵢ flows in the helix, that is, an alternating current having an angular frequency ω. A turn is intended to mean a winding of all four wires. The turn difference of a turn of this type is approximately 0.13 mm at 4.105% compression, which equals 0.546 mm at approximately 5% compression. A sleeve that is comprised, e.g. of MP35N®, and has the mean diameter is provided on the far outside, and through which current Iₐ induced by the inner helix flows. This sleeve is electrically insulated against the inner helix. The current flow is generated in that the sleeve also encloses the surface of the inner helix, thereby coupling the two in an inductive manner.

The inductance that is generated by the current flowing in the inner helix and occurs only in the surface of the inner helix is

$${B}_{i} = μ \frac{{I}_{i} \cdot N}{l} .$$

In that expression, N is the number of loops that extend for distance l, which is covered side-by-side with sleeves, l>>dₐ. The inductance generated by the current on the sleeve is

$${B}_{a} = μ \frac{{I}_{a}}{l} .$$

To determine the magnetic flux that passes through the sleeve, multiply the inductances by the cross-sectional areas that enclose the currents that generate them, according to:

$${Φ}_{ges} = {Φ}_{i} + {Φ}_{a} = \frac{π \cdot {d}_{i}^{2}}{4} \cdot μ \cdot \frac{{I}_{i} \cdot N}{l} + \frac{π \cdot {d}_{a}^{2}}{4} \cdot μ \cdot \frac{{I}_{a}}{l} = {L}_{i} \cdot {I}_{i} + {L}_{a} \cdot {I}_{a}$$

in which

$${L}_{i} := \frac{μ \cdot π \cdot N \cdot {d}_{i}^{2}}{4 \cdot l} and {L}_{a} := \frac{μ \cdot π \cdot {d}_{a}^{2}}{4 \cdot l} .$$

The sleeve is a closed circuit, and therefore the voltages sum to zero. The voltages on the sleeve are comprised of the induced voltage of the inner helix, defined as jωIᵢLᵢ the self-inductance of the sleeve, defined as jωIₐLₐ, and the voltage drop across the resistance of the sleeve, defined as IᵢRₐ. The following must therefore apply jωIᵢLᵢ+jωIₐLₐ+IₐRₐ=0, and the following applies for the induced current in the sleeve

$${I}_{a} = \frac{jω {L}_{i}}{{R}_{a} + j ω {L}_{a}} {I}_{i} .$$

To determine the effect of the sleeves, or inductive rings, on the inner helix, the effect of the induced current Iₐ on the inner helix must be investigated. The same loop rule used for the sleeve will now therefore be applied to the inner helix, wherein Uᵢ is the voltage applied to the ends of the electrode lead or helix, and is determined as follows:

$$\begin{matrix}{U}_{i} = {I}_{i} {R}_{i} + j ω {I}_{i} {L}_{i} N + jω \frac{\frac{π}{4} {d}_{i}^{2}}{\frac{π}{4} {d}_{a}^{2}} {NL}_{a} {I}_{a} \\ = {I}_{i} {R}_{i} + j ω {I}_{i} {L}_{i} N + jω \frac{{d}_{i}^{2}}{{d}_{a}^{2}} {NL}_{a} \frac{jω {L}_{i}}{{R}_{a} + jω {L}_{a}} {I}_{i}\end{matrix}$$

Taken into account herein is the fact that only a portion Φₐ of the magnetic flux

$$\frac{\frac{π}{4} {d}_{i}^{2}}{\frac{π}{4} {d}_{a}^{2}} {Φ}_{a}$$

generated by the sleeve also penetrates the surface

$$\frac{π}{4} {d}_{i}^{2}$$

of the helix. In addition, the magnetic flux passes through all turns in the helix and must therefore be multiplied by N. Separating the real part and the imaginary part, that is, the effective resistance and the reactance, results in the expression for the impedance of the helix:

$${Z}_{i} = \frac{{U}_{i}}{{I}_{i}} = {R}_{i} + \frac{{ω}^{2} {NL}_{i} {L}_{a}}{{R}_{a}^{2} + {ω}^{2} {L}_{a}^{2}} \frac{{d}_{i}^{2}}{{d}_{a}^{2}} + j ω {L}_{i} N ( 1 - \frac{{d}_{i}^{2}}{{d}_{a}^{2}} \frac{{ω}^{2} {L}_{a}^{2}}{{R}_{a}^{2} + {ω}^{2} {L}_{a}^{2}} )$$

Dividing all of this by length l, and therefore defining Zᵢ′, Lᵢ′=N′Lᵢ and Rᵢ′ as resistances and inductances per unit of length, the result is

$${Z}_{i}^{′} = {R}_{i}^{′} + \frac{{ω}^{2} {L}_{i}^{′} {L}_{a}}{{R}_{a}^{2} + {ω}^{2} {L}_{a}^{2}} \frac{{d}_{i}^{2}}{{d}_{a}^{2}} + j ω {L}_{i}^{′} ( 1 - \frac{{d}_{i}^{2}}{{d}_{a}^{2}} \frac{{ω}^{2} {L}_{a}^{2}}{{R}_{a}^{2} + {ω}^{2} {L}_{a}^{2}} ) .$$

The resistance of the inner helix given direct current Rᵢ′ is therefore also joined by a frequency-dependent part

$$\frac{{ω}^{2} {L}_{i}^{′} {L}_{a}}{{R}_{a}^{2} + {ω}^{2} {L}_{a}^{2}} \frac{{d}_{i}^{2}}{{d}_{a}^{2}} .$$

Wavelength k along the lead depends on the values for inductance, capacitance, and resistance per unit length, k²=ω²C′L′−G′R′−jω(C′ R′+L′ G′), wherein G′ is the conductance of the insulating tube and, assuming it is a perfect insulator, is set approximately to zero. This leaves k=\sqrt{ω²C′L′−jωC′R′}=:β+jα, wherein β is the real part of the wave number and describes the wavelength

$$( λ = \frac{2 π}{β} ) ,$$

while α is the damping constant of the wave on the conductor.

$$α = \sqrt{\frac{\sqrt{{( {ω}^{2} {L}^{′} {C}^{′} )}^{2} + {( ω {R}^{′} {C}^{′} )}^{2}} - {ω}^{2} {L}^{′} {C}^{′}}{2}}$$

is ω²C′L′>ωC′R′. If the values for Zᵢ′ and

$${R}^{′} = {R}_{i}^{′} + \frac{{ω}^{2} {L}_{i}^{′} {L}_{a}}{{R}_{a}^{2} + {ω}^{2} {L}_{a}^{2}} \frac{{d}_{i}^{2}}{{d}_{a}^{2}}$$

from the formula for the impedance of the helix

$${L}^{′} = {L}_{i}^{′} ( 1 - \frac{{d}_{i}^{2}}{{d}_{a}^{2}} \frac{{ω}^{2} {L}_{a}^{2}}{{R}_{a}^{2} + {ω}^{2} {L}_{a}^{2}} )$$

are used here, the result is the optimal value R_{a,opt} which yields the greatest damping, with R_{a,opt}≈ωLₐ.

For a four-fold inner helix having a mean diameter of 0.57 mm and a wire diameter of 0.13 mm, one obtains L′≈1.07 μH/m. Realistically, sleeves having a mean diameter of dₐ=0.77 mm could be slid over them. If they were comprised of MP35N®, the optimal wall thickness would be 10.63 μm. In the case in which the distances between the sleeves are as long as the sleeves, the wall thickness of the sleeves must be doubled in order to obtain the optimal resistance value once more, averaged by the length of the electrode in meters.

Moreover, the distances separating the rings or sleeves must be substantially smaller than their diameter dₐ. These components, which are described as sleeves, can also be present in the form of closed wire loops. Most importantly, they are arranged side-by-side in a row and form closed loops having optimal resistance, in order to achieve strong damping of high-frequency waves.

Given a wall thickness of the sleeves of 10.63 μm, then, in this example, a resistance of 220 Ω/m would be added to the resistance of the helix of 66 Ω/m, at 64 MHz. The inductance of the helix would then be only 0.777 μH/m, at 64 MHz. Given a capacitance per unit length of 160 pF/m, the damping constant without rings is αₒₕₙₑ=0.402 Np/m, and with rings is αₘᵢₜ=1.88 Np/m. Assuming that electrical energy is coupled in evenly along the electrode and is transmitted to the electrode tip, the energy, in particular, that enters the helix close to the proximal end is damped more heavily toward the distal end.

Clearly, under these conditions, the current can be reduced by 30% and the energy in the tip can be reduced by 50% for an electrode having a length of 60 cm.

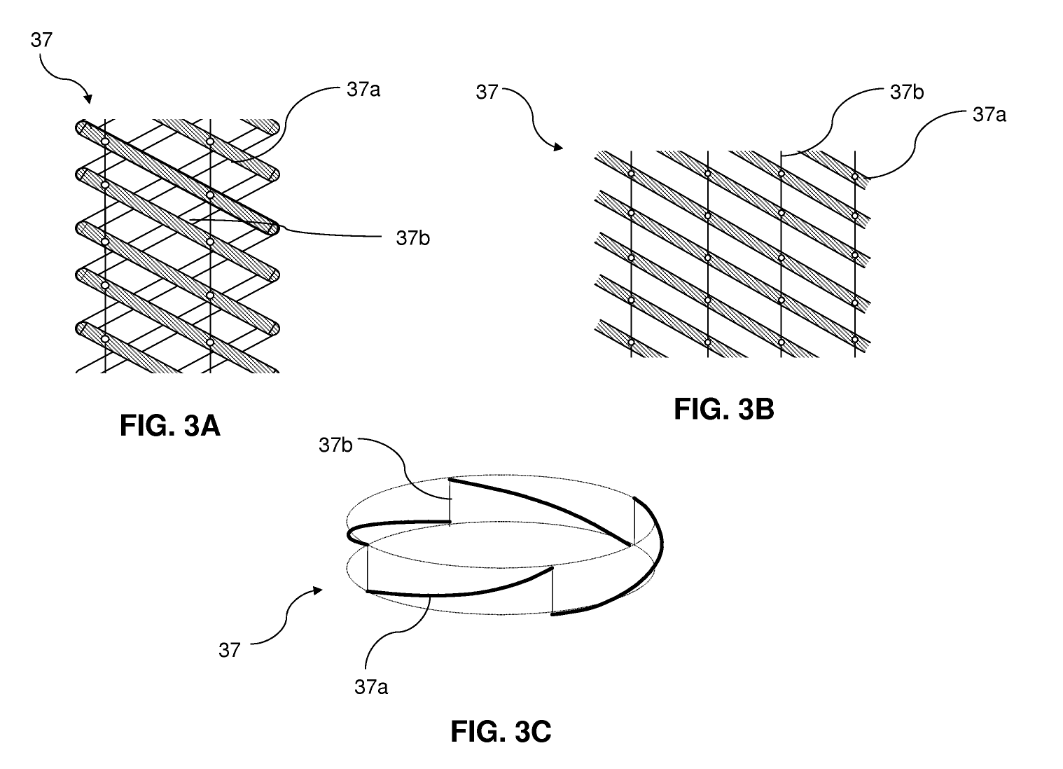

FIGS. 3A-3C show, as sketches of a side view (FIG. 3A) respectively, the plane of a winding (FIG. 3B), and a perspective sectional view (FIG. 3C), of a further embodiment of inductive resistive circuit elements 7 of a implantable lead according to the invention. In this case, inductive resistive circuit elements 37 include a spiral-wound strip, or a wire, 37a, which is a subsection of the aforementioned helical functional lead, and connecting wires 37b which conductively interconnect the individual turns of helix 37a in the longitudinal direction of the implantable lead. Connecting wires 37b can be welded to the helix 37a, or be bonded or soldered thereto in a conductive manner.

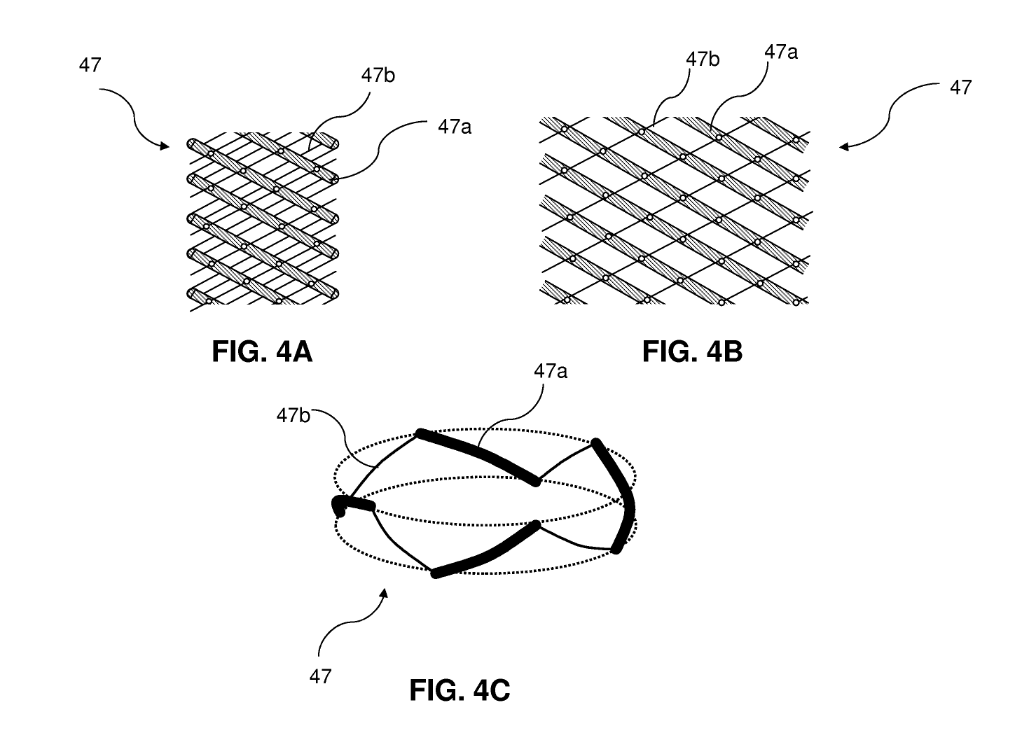

FIGS. 4A-4C show, as a modified embodiment and in a manner that corresponds to the depiction in FIGS. 3A-3C, field-coupling lead elements 7 and 47 which, in turn, comprise a strip, or wire, helix 47a and connecting wires 47b as described above. In contrast to the aforementioned embodiment, in this case, the connecting wires do not extend in the longitudinal direction of the lead but, rather, obliquely thereto. This makes it substantially easier to deform the lead, in which case the oblique wires then change their local angle of inclination, while longitudinally extending connecting wires oppose deformation with considerable resistance. To further simplify the deformation, it can also be provided that, in contrast to the above-described configuration, the helix which forms the connecting wires are not attached via welding, nor are they conductively bonded or soldered thereto. Instead, the helix with the connecting wires is “crimped” to the functional-lead helix, e.g., by designing the two helixes to have the same inner diameter and mounting the helix with the connecting helixes externally onto the functional-lead helix.

FIGS. 3C and 4C each show how the current induced in the inductive resistive circuit element by the external alternating magnetic field forms a circuit element in a segment covering, in each case, four quarter turns of helix 37a and 47a and connecting wires 37b and 47b. Since the circuit element (or the integrated inductive ring formed as a result) shares almost the same enclosed surface area as the associated electrode helix (i.e., the functional lead), the inductive coupling is greater than it is for the above-described induction rings and sleeves shown in FIGS. 1 and 2A and 2B.

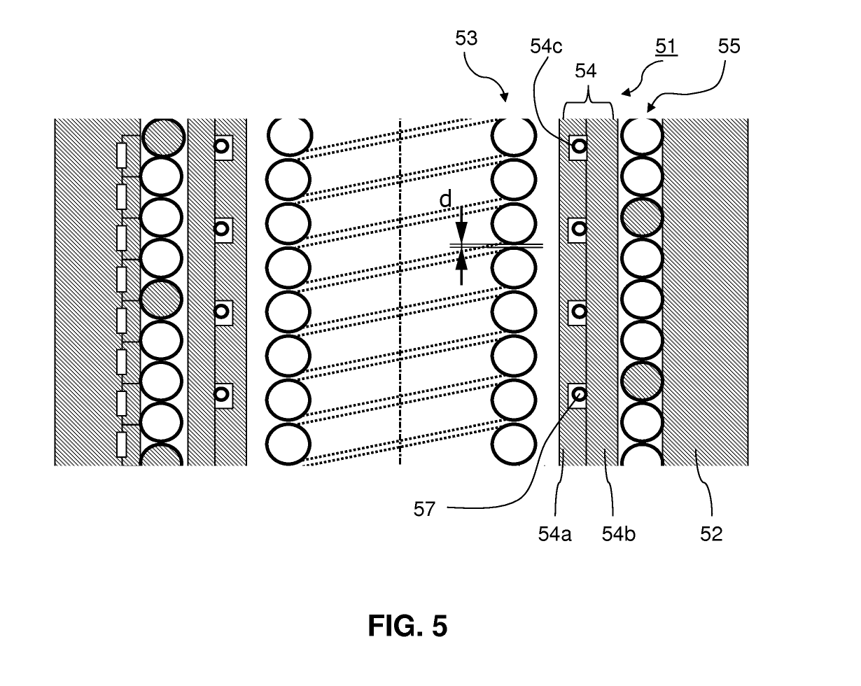

FIG. 5 shows, in a schematic cross-sectional depiction and as a further embodiment, a lead 51 according to the invention, in the case of which an inner helix 53, which is wound from two individual wires to form a lead body 52, and an outer helix 55, which is wound from four individual wires, are embedded in the lead body 52. An insulation 54 comprised of two concentric cylinders 54a, 54b is provided between the inner helix 53 and the outer helix 55. Inner insulation material 54a of this insulation comprises grooves or channels 54c, which are formed on the outer side, and into which wire rings 57 are placed, as induction rings in the sense of the schematic diagram shown in FIG. 1. The diameter ratio between wire rings 55 and the wires of inner 53 and outer 55 helixes is intended to show that the wire diameter of the induction rings is substantially smaller than that of the functional lead.

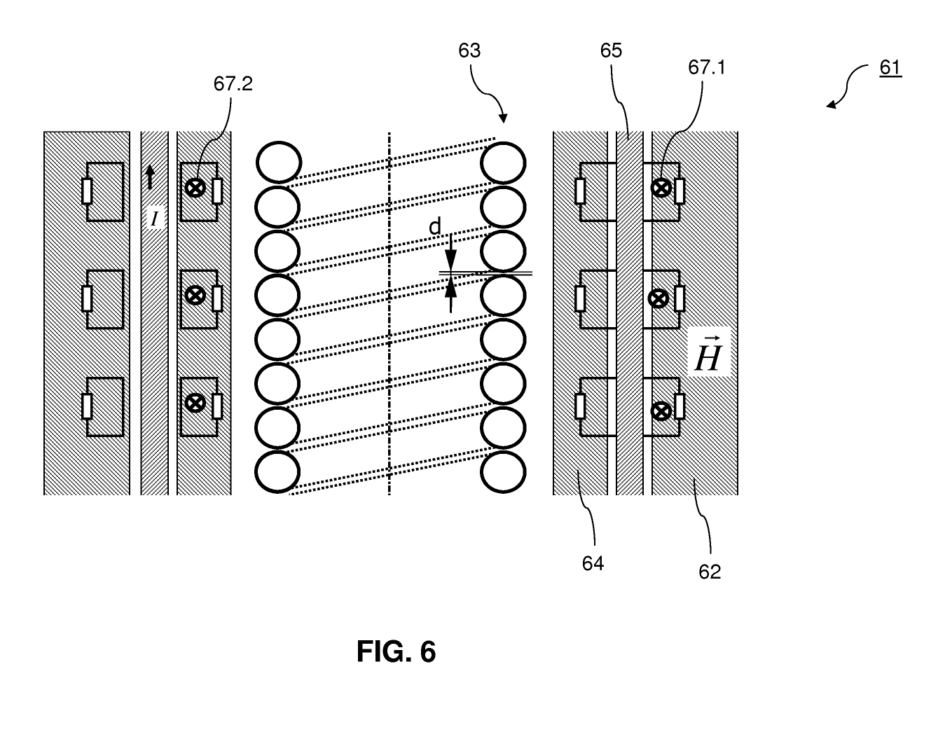

FIG. 6 shows, in a synergistic depiction and as modified embodiment(s), a further electrode lead 61 comprising a lead body 62, a middle insulation 64, and inner conductor structure 63 of which corresponds to the embodiment shown in FIG. 5, and which has a cord structure 65 instead of a four-fold helix as the outer conductor. Field-decoupling rings (induction rings) 67.1 are shown on the right side of FIG. 6, and they are placed in the inner surface of outer lead body 62 in a manner such that they have resistance-laden, electrical contact with outer conductor 65, while inductive resistive circuit elements 67.2 are depicted symbolically on the left side of FIG. 6, which are embedded in middle insulation 64 without electrical contact to outer conductor 65.

In FIGS. 5 and 6, the symbol for electrical resistance represents a wire with resistance per unit length, and silicone is assumed to be the material of the lead body and the intermediate insulation. The specific dimensions shown in the two figures are intended merely to represent examples of dimensions for marketable electrode leads that are to be improved using the means according to the invention, and are in no way meant to be limiting.

The embodiments of the invention are not limited to the above-described examples and emphasized aspects, but rather are possible in a large number of modifications that lie within the scope of a person skilled in the art.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. An implantable lead comprising:
an elongate lead body; and
a functional lead which extends in a longitudinal direction of the elongate lead body and enables the implementation of a medical function of the lead, wherein, in addition to the functional lead and insulated therefrom, a plurality of inductive resistance circuit elements are embedded in the lead body, which reduce a coupling of the functional lead with an external alternating magnetic field or increase the damping of electrical high-frequency energy transmitted along the lead.

2. The implantable lead according to claim 1, wherein the plurality of inductive resistance circuit elements are designed as rings or ring segments which are disposed in a row and interspaced in the longitudinal direction of the elongate lead body.

3. The implantable lead according to claim 1, wherein the plurality of inductive resistance circuit elements are designed as wire loops or windings which are disposed in a row and interspaced in the longitudinal direction of the lead body.

4. The implantable lead according to claim 1, wherein the plurality of inductive resistance circuit elements are distributed evenly along the length of the elongate lead body and are disposed equidistantly from each other.

5. The implantable lead according to claim 4, wherein the distances between the inductive resistance circuit elements are smaller than their diameter.

6. The implantable lead according to claim 1, wherein the plurality of inductive resistance circuit elements are placed in preformed recesses in the elongate lead body.

7. The implantable lead according to claim 1, further comprising a first and a second functional lead which extend coaxially relative to one another, wherein the plurality of inductive resistance circuit elements are disposed in a radial direction between the first and the second functional leads and are insulated from the first and/or the second functional leads.

8. The implantable lead according to claim 1, wherein a helix of the functional lead that extends in the longitudinal direction of the elongate lead body, the turns of which do not have contact with each other, and longitudinal wires that electrically interconnect the turns along the length of the elongate lead body and extend in the direction of the longitudinal axis of the elongate lead body, are provided as the inductive resistance circuit elements.

9. The implantable lead according to claim 8, wherein the helix of the functional lead is designed as a strip or a wire.

10. The implantable lead according to claim 1, wherein a helix of the functional lead that extends in the longitudinal direction of the elongate lead body, the turns of which do not have contact with each other, and connecting wires which are slanted relative to the longitudinal axis of the elongate lead body and which extend along at least a subsection of the circumference thereof and electrically connect at least two adjacent turns of the helix, are provided as the inductive resistance circuit elements.

11. The implantable lead according to claim 10, wherein the connecting wires are subsections of a wire helix that extends contradirectionally to the helix of the functional lead.

12. The implantable lead according to claim 10, wherein the helix of the functional lead is designed as a strip or a wire.

13. The implantable lead according to claim 1, wherein the field-decoupling lead elements include a nickel-cobalt alloy.

14. The implantable lead according to claim 13, wherein the nickel-cobalt alloy comprises MP35N®.
